(19) United States
(12) Plant Patent Application Publication
(10) Pub. No.: US 2005/0034202 P1
(43) Pub. Date: Feb. 10, 2005

(54) Rhododendron obtusum 'Melle'

(76) Inventors: Lieven De Waele, Oostakker (BE); Magda De Baerdemaeker, Oostakker (BE)

Correspondence Address: AGIER & ASSOCIATES INTERNATIONAL, 32 Avenue du General de Gaulle, VAUJOURS, 93410, FR

(21) Appl. No.: 10/877,200

(22) Filed: Jun. 28, 2004

(30) Foreign Application Priority Data
Jun. 13, 2000 (DE) RDG 188

(51) Int. Cl. A01H 5/00

(52) U.S. Cl. PLT/226

(57) ABSTRACT

A new and distinct variety of Azalea plant named ‘Melle’, characterized by its unusually attractive clusters of red purple white margined double hose-in-hose larger than normal bi-colored blossoms with darker blotches at the center of corolla, which retain their beautiful appearance even in inclement weather. When compared to its peers this new cultivar has proven to be a very late bloomer. It forms an upright and outward spreading vigorous shrub with a freely branching habit and medium green foliage on a plentiful basis that turns to and remains bright orange until late winter. It has nonetheless exhibited excellent heat and drought tolerance.

### BACKGROUND OF INVENTION

The present invention relates to a new and distinct cultivar of Azalea, botanically known as Rhododendron obtusum and hereinafter referred to by the cultivar name ‘Melle’. This new Azalea cultivar was discovered by the Inventor in Oostakker, Belguim, as a naturally occurring mutation of the non patented Rhododendron Obtusum ‘Rosa’. The new Azalea variety occurred as a single plant within a population of the here above mentioned parent cultivar. This new variety of Azalea obtuse was selected with regard to its many differences in flower colors.

Asexual reproduction of the new cultivar ‘Melle’ by terminal cuttings taken in various locations both in Europe and the US has shown that the unique characteristics of this new Azalea plant are stable and reproduced true to type successively, from one generation to another.

### BRIEF DESCRIPTION OF INVENTION

Plants of the new Azalea cultivar named ‘Melle’ have not been observed under all possible environmental conditions. The phenotype may somewhat vary with fluctuations in environment such as light intensity, day length, temperature and fertility levels without however any variation in genotype.
1. Forms sturdy and unusually attractive clusters of red purple white margined double hose-in-hose larger than normal bi-colored blossoms with darker blotches at the center.
2. Forms upright and outward spreading vigorous shrub.
3. Exhibit's a freely branching habit.
4. Forms medium green foliage on a plentiful basis that turns to and remains bright orange until late winter.
5. Has proven through testing in various locations to be a very late bloomer when compared to its peers.
6. Exhibits excellent heat tolerance.
7. Exhibits drought tolerance.
8. Has achieved a nearly 100% take rate through terminal cuttings propagation.

Substantially as herein shown and described.

In a side-by-side comparisons conducted by the Inventor in Oostakker, Belgium and by the Assignee in Vaujours, France, it appears that plants of the new Azalea cultivar differ from their parent cultivar, Rhododendron Obtusum ‘Rosa’ by the following features:
1. Bears unusually attractive clusters of red purple white margined double hose-in-hose bicolored blossoms with darker blotches at the center.
2. Has proven through testing in various locations to be a later bloomer than its parent cultivar.

### BRIEF DESCRIPTION OF THE PHOTOGRAPHS

This new azalea hybrid variety is illustrated by the accompanying photographic prints in which:

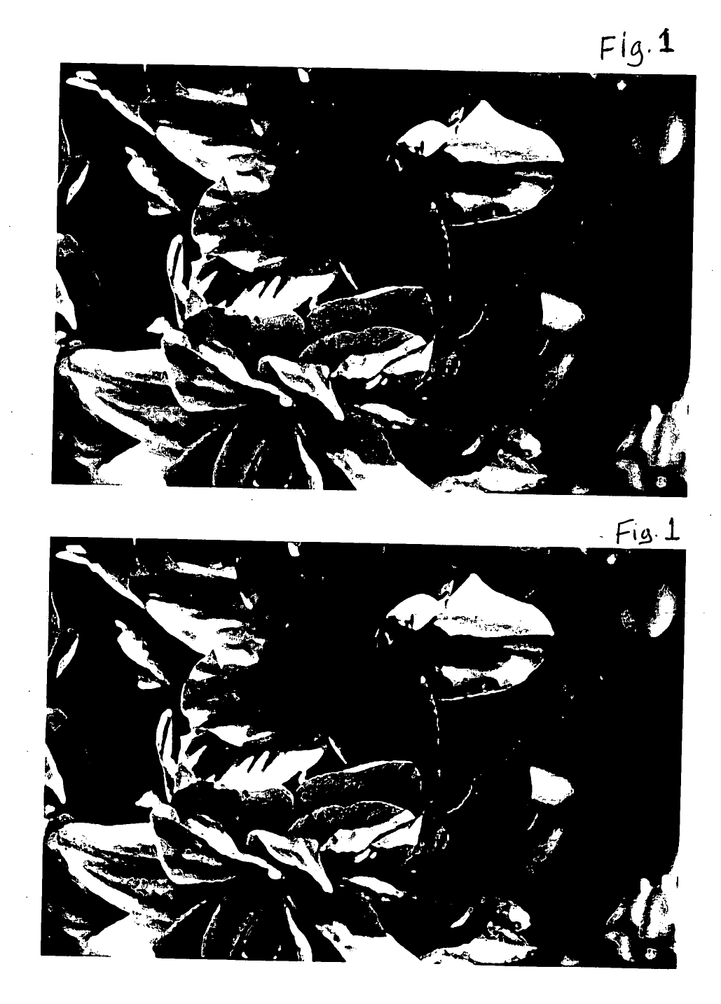

1. FIG. 1 is a close-up showing flower color and flower form.

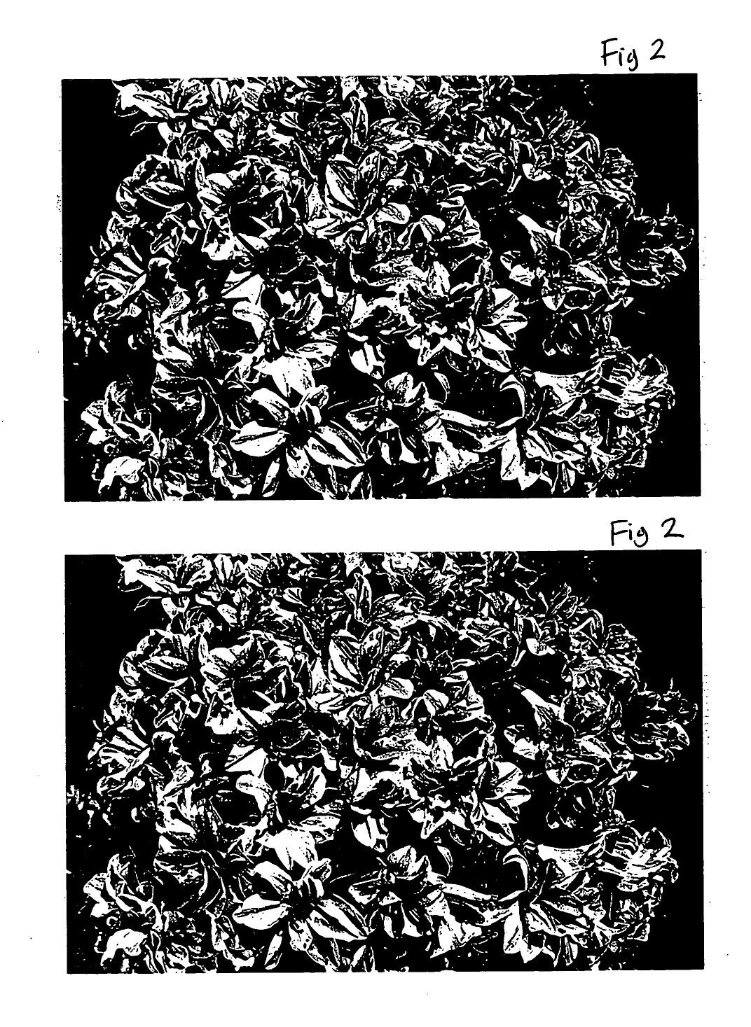

2. FIG. 2 shows growth habit and heavy blooming qualities.

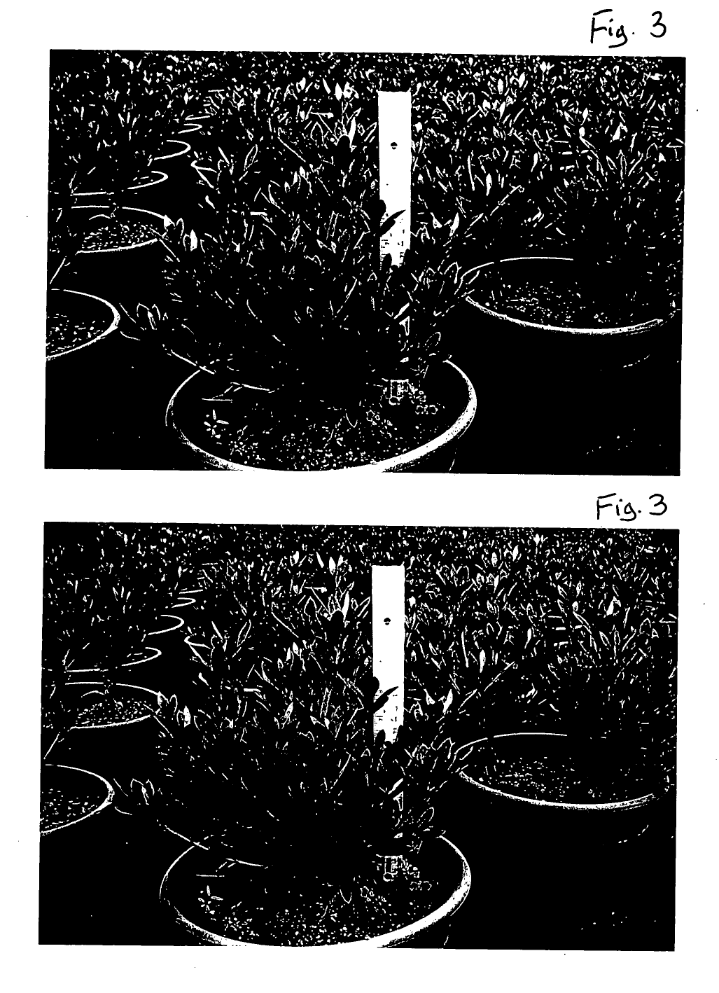

3. FIG. 3 shows a young plant, 11(eleven) months after rooting in May 2004.

The colors shown are as true as is reasonably possible to obtain by conventional photographic procedures. The colors of the various plant parts are defined with reference to The Royal Horticultural Society Color Chart. Description of colors in ordinary terms are presented where appropriate for clarity in meaning.

### DETAILED BOTANICAL DESCRIPTION

Botanical name of taxon: Rhododendron obtusum.
Common name of taxon: Japanese Azalea.
Variety denomination: Melle.
Foliage: Foliage exhibit's the same characteristics as parent cultivar.
Young leaf: Color of upper side is medium green.
Mature leaf:
   Length.—Long.
   Width.—Medium.
   Shape.—Elliptic.
   Color of upper side.—Dark green.
   Color of lower.—Medium green.
   Shape of apex.—Mucronate.
Flowers: Forms sturdy and unusually attractive clusters of red purple white margined double hose-in-hose larger than normal bicolored blossoms with darker blotches at the center.
Pedicel length: Medium to long.
Calyx: Present.
Calyx formation of a corolla form: Absent or very weak.
Flower:
   Diameter.—Large.
   Shape.—Funnel-shaped.
   Fragrance.—Absent to very weak.
   Flower.—Type of corolla: double.
   Corolla lobe.—Color of margin of upper side: white RHS 155D.
   Corolla lobe.—Color of middle of upper side: red-purple RHS 57 C/D.
   Corolla lobe.—Color of middle of lower side: red-purple RHS 57 D.
   Corolla lobe.—Undulation of margin: absent to very weak.
   Flower throat.—Conspicuousness of markings: strong.
   Flower throat.—Type of markings: spots touching each other.
   Flower throat.—Color of markings: red-purple RHS 60 A/B.
   Flower throat.—Color compared to color of middle of upper side of corolla lobe: lighter.
   Time of flowering.—Very late.

1. I claim a new and distinct variety of Azalea plant named ‘Melle’ as illustrated and characterized by the following combination of traits which have repeatedly been observed and determined as being the unique features of this new variety of Azalea.
